(19) United States
(12) Patent Application Publication
(10) Pub. No.: US 2026/0345263 A1
(43) Pub. Date: Nov. 5, 2026

(54) SURGICAL ROBOTIC SYSTEM AND METHOD FOR PREVENTING INSTRUMENT COLLISION

(71) Applicant: Covidien LP

(72) Inventors: Connor D. Roberts, Arvada, CO (US); Christopher T. Tschudy, Arvada, CO (US); Amando J. Vigil, Northglenn, CO (US)

(21) Appl. No.: 19/168,520

(22) PCT Filed: Mar. 20, 2024
(86) PCT No.: PCT/IB2024/052686

§ 371 Date: Sep. 24, 2025

Related U.S. Application Data
(60) Provisional application No. 63/456,051, filed on Mar. 31, 2023.

(51) Int. Cl. A61B 34/32; A61B 34/20

(52) U.S. Cl. CPC A61B 34/32; A61B 34/20; A61B 2034/2055; A61B 2034/2065

(57) ABSTRACT

A surgical robotic system includes a first robotic arm having a first robotic instrument and a second robotic arm having a second robotic instrument. The system also includes a camera configured to capture a video stream of the first robotic instrument and the second robotic instrument. The system further includes a video processing device coupled to the camera and configured to identify the first robotic instrument and the second robotic instrument in at least one frame of the video stream. The video processing device is also configured to generate a first boundary around a portion of the first robotic instrument and a second boundary around a portion of the second robotic instrument. The video processing device is further configured to calculate an overlap between the first boundary and the second boundary. The system also includes a controller configured to predict a collision based on the overlap between the first boundary and the second boundary and to adjust at least one parameter of the first robotic arm or the second robotic arm in response to the prediction of the collision.

### CROSS-REFERENCE TO RELATED APPLICATION

This application claims the benefit of U.S. Provisional Patent Application Ser. No. 63/456,051, filed Mar. 31, 2023, the entire content of which is incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. The surgical robotic systems are extremely complex and use a variety of safety features monitoring their operation.

### SUMMARY

According to one embodiment of the present disclosure, a surgical robotic system is disclosed. The surgical robotic system includes a first robotic arm having a first robotic instrument and a second robotic arm having a second robotic instrument. The system also includes a camera configured to capture a video stream of the first robotic instrument and the second robotic instrument. The system further includes a video processing device coupled to the camera and configured to identify the first robotic instrument and the second robotic instrument in at least one frame of the video stream. The video processing device is also configured to generate a first boundary around a portion of the first robotic instrument and a second boundary around a portion of the second robotic instrument. The video processing device is further configured to calculate an overlap between the first boundary and the second boundary. The system also includes a controller configured to predict a collision based on the overlap between the first boundary and the second boundary and to adjust at least one parameter of the first robotic arm or the second robotic arm in response to the prediction of the collision.

Implementations of the above embodiment may include one or more of the following features. According to one aspect of the above embodiment, each of the first boundary and the second boundary may be a 3D boundary volume. The 3D boundary volume may include a plurality of nested volumes. Each of the first boundary and the second boundary may be a 2D shape. The parameter may be one of a torque limit or a speed limit of an actuator of the first robotic arm or the second robotic arm. The surgical robotic system may also include a screen configured to display a graphical user interface (GUI). The controller may be further configured to output a prompt on the GUI in response to the prediction of the collision.

According to another embodiment of the present disclosure, a surgical robotic system is disclosed. The surgical robotic system includes a first robotic arm having a first robotic instrument and a second robotic arm having a second robotic instrument. The system also includes a camera configured to capture a video stream of the first robotic instrument and the second robotic instrument, and a video processing device coupled to the camera and configured to identify the first robotic instrument and the second robotic instrument in at least one frame of the video stream. The video processing device is further configured to calculate an angle between the first robotic instrument and the second robotic instrument. The system also includes a controller configured to compare the calculated angle to an angle threshold and predict a collision based on the calculated angle being lower than the angle threshold. The controller is further configured to adjust at least one parameter of the first robotic arm or the second robotic arm in response to the prediction of the collision.

Implementations of the above embodiment may include one or more of the following features. According to one aspect of the above embodiment, the first robotic arm may include a first marker and the second robotic arm may include a second marker. The surgical robotic system may also include a tracking unit configured to detect locations of the first marker and the second marker. The controller may be further configured to calculate a distance between the first robotic arm and the second robotic arm based on the locations of the first marker and the second marker. The controller may be additionally configured to compare the calculated distance to a distance threshold. The controller may be also configured to predict the collision based on the calculated distance being lower than the distance threshold. The surgical robotic system may include a surgeon console having at least one hand controller configured to receive user input for controlling at least one of the first robotic arm or the second robotic arm. The controller may be further configured to calculate a pose for each of the first robotic arm and the second robotic arm based on the user input and to determine whether the calculated pose of the first robotic arm and the second robotic arm would result in a collision. The surgical robotic system may also include a screen configured to display a graphical user interface (GUI). The controller may be further configured to output a prompt on the GUI in response to the prediction of the collision.

According to a further embodiment of the present disclosure, a method for predicting a collision between robotic instruments is disclosed. The method includes capturing, through a laparoscopic camera, a video stream of a first robotic instrument of a first robotic arm and a second robotic instrument of a second robotic arm. The method also includes identifying, at a video processing device, the first robotic instrument and the second robotic instrument in at least one frame of the video stream, generating a first boundary around a portion of the first robotic instrument and a second boundary around a portion of the second robotic instrument. The method further includes calculating an overlap between the first boundary and the second boundary and predicting a collision based on the overlap between the first boundary and the second boundary. The method additionally includes adjusting at least one parameter of the first robotic arm or the second robotic arm in response to the prediction of the collision.

Implementations of the above embodiment may include one or more of the following features. According to one aspect of the above embodiment, the method may also include displaying, on a screen, a graphical user interface (GUI) and outputting a prompt on the GUI in response to prediction of the collision.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a schematic diagram of a system for determining phases of a surgical procedure according to an embodiment of the present disclosure;

FIG. 7 is a flow chart of a method for monitoring instrument collisions according to an embodiment of the present disclosure;

FIG. 8 is a flow chart of a method for preventing instrument collisions according to an embodiment of the present disclosure;

FIG. 9 is a laparoscopic image of an instrument including an augmented boundary volume and other markers according to an embodiment of the present disclosure;

FIG. 10 is a schematic diagram of a pair of instruments with boundary volumes according to an embodiment of the present disclosure;

FIG. 11 is a laparoscopic image of an instrument including an augmented boundary box according to an embodiment of the present disclosure;

FIG. 12 is a schematic diagram of a pair of instruments with a boundary box according to an embodiment of the present disclosure;

FIG. 13 is a flow chart of a method for preventing instrument or robotic arm collisions according to an embodiment of the present disclosure; and

FIG. 14 is a schematic diagram of a pair of instruments disposed at an angle of concern according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

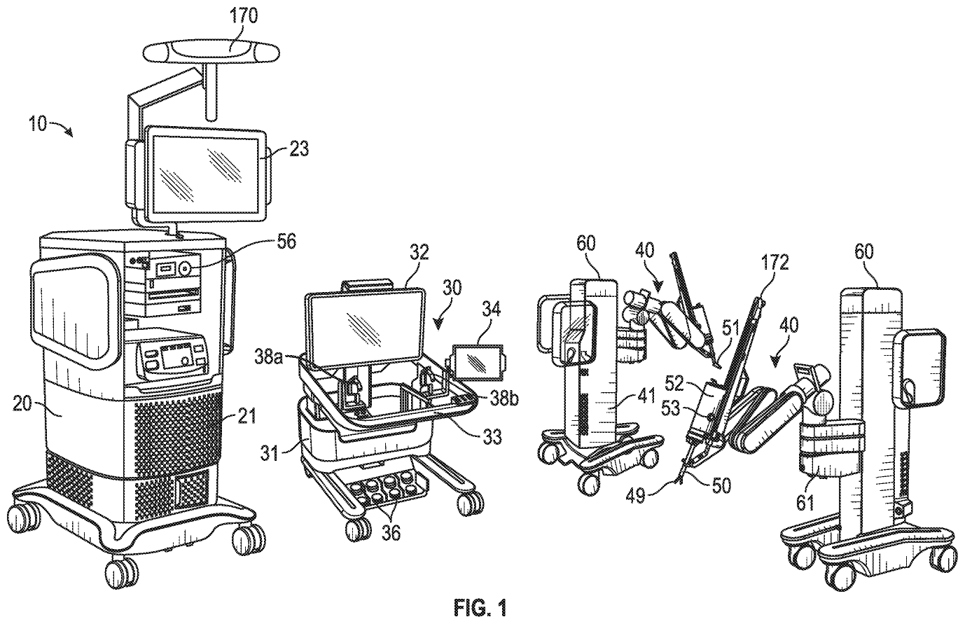

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arms 40 also couple to the mobile carts 60. The robotic system 10 may include any number of mobile carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include a laparoscopic camera 51 configured to capture video of the surgical site. The laparoscopic camera 51 may be a stereoscopic endoscope configured to capture two side-by-side (i.e., left and right) images of the surgical site to produce a video stream of the surgical scene. The laparoscopic camera 51 is coupled to an image processing device 56, which may be disposed within the control tower 20. The image processing device 56 may be any computing device configured to receive the video feed from the laparoscopic camera 51 and output the processed video stream.

The surgeon console 30 includes a first screen 32, which displays a video feed of the surgical site provided by camera 51 of the surgical instrument 50 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

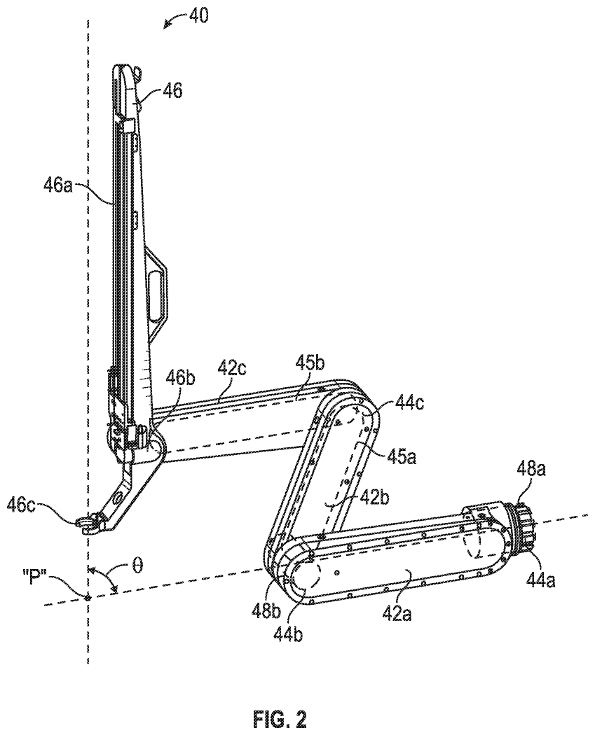

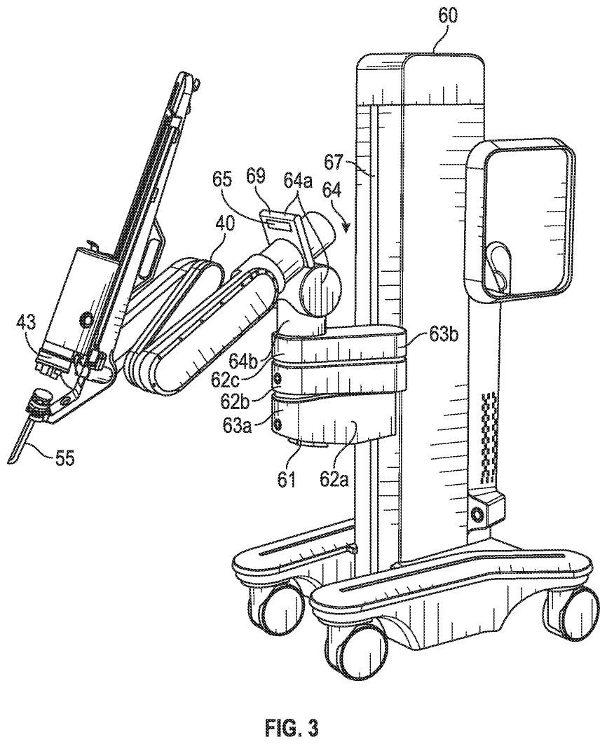

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle 0 between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

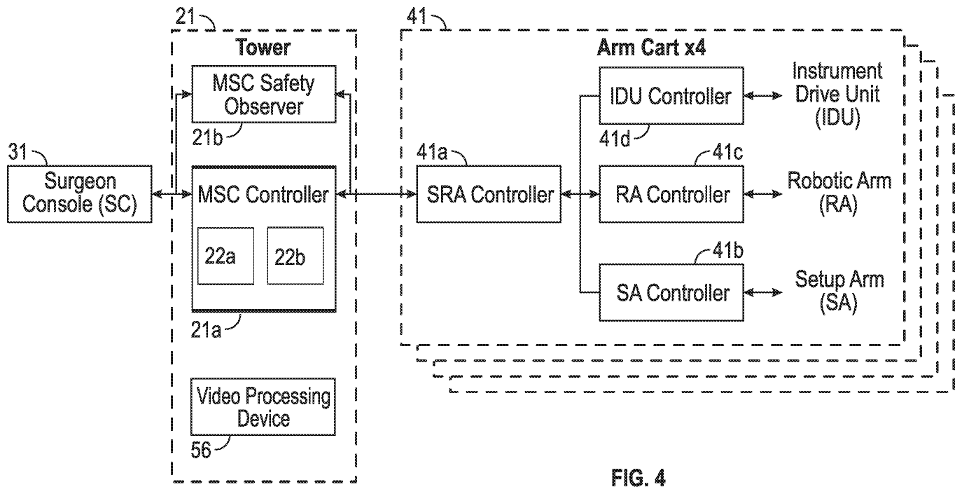

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the hand controller 38a is then scaled by a scaling function executed by the controller 21a. In embodiments, the coordinate position may be scaled down and the orientation may be scaled up by the scaling function. In addition, the controller 21a may also execute a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

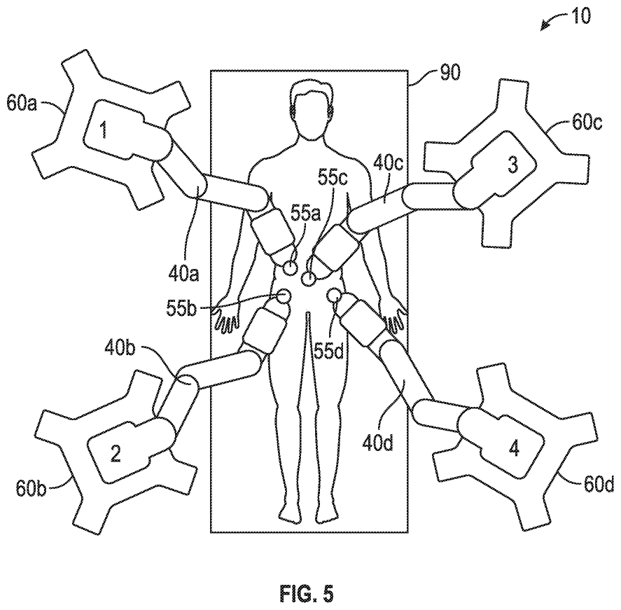

With reference to FIG. 5, the surgical robotic system 10 is setup around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

A surgical procedure may include multiple phases, and each phase may include one or more surgical actions. As used herein, the term “phase” represents a surgical event that is composed of a series of steps (e.g., closure). A “surgical action” may include an incision, a compression, a stapling, a clipping, a suturing, a cauterization, a sealing, or any other such actions performed to complete a phase in the surgical procedure. A “step” refers to the completion of a named surgical objective (e.g., hemostasis). During each step, certain surgical instruments 50 (e.g., forceps) are used to achieve a specific objective by performing one or more surgical actions.

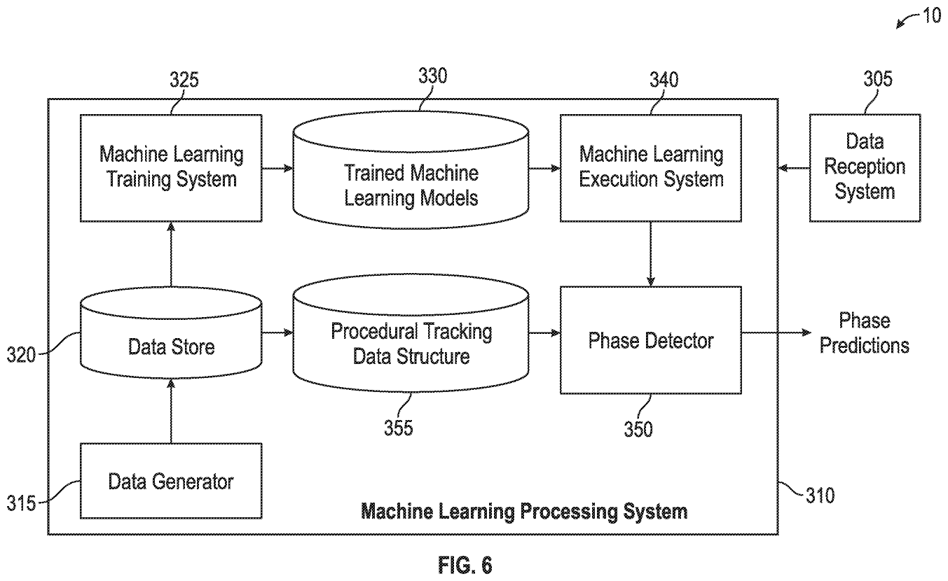

With reference to FIG. 6, the surgical robotic system 10 may include a machine learning (ML) processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual images, or record the video data from the video processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure (“procedure”). Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine when to implement the methods described below.

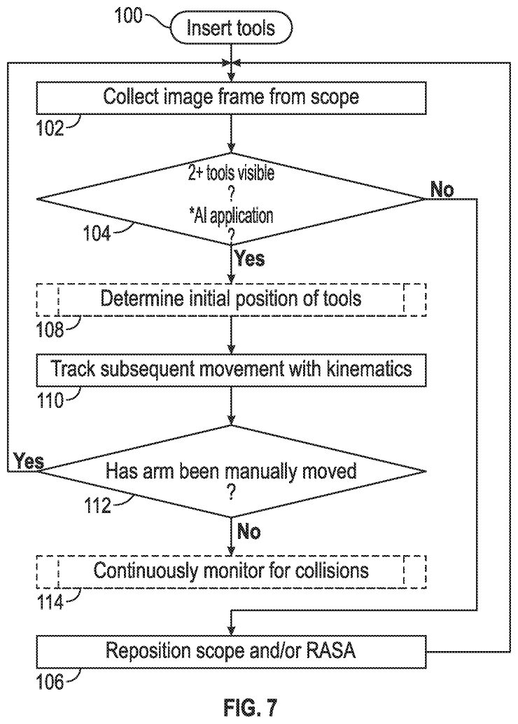

With reference to FIG. 7, a method for predicting and preventing instrument collision includes insertion of instruments 50 at step 100 into the patient as described above with respect to FIG. 5. The method of FIG. 7, in part or in whole, may be embodied as software instructions executable by a processor, e.g., controller 21a. At step 102, camera 51 is used to capture images (e.g., video stream) of the instruments 50. The individual frames of the video stream are processed at the video processing device 56 using any suitable computer vision algorithm suitable for identifying objects, e.g., machine learning algorithms trained on data including images of instruments. Instrument 50 includes a fiducial marker 150 (FIG. 9) having a plurality of geometric shapes identifiable by the computer vision algorithm executed by the video processing device 56. The fiducial marker 150 allows the video processing device 56 to calculate the shape, size, orientation, position, and other physical parameters of the instrument 50 in relation to the surgical site and other objects captured by the camera 51. The fiducial marker 150 may be detected in one or more a plurality of frames of the video stream. The video processing device 56 may collect and process a sufficient number of frames, e.g., 16 or more, until the fiducial marker 150 is identified and in each of the sampled frames to allow the video processing device 56 to track the fiducial marker 150 across all frames of the video stream.

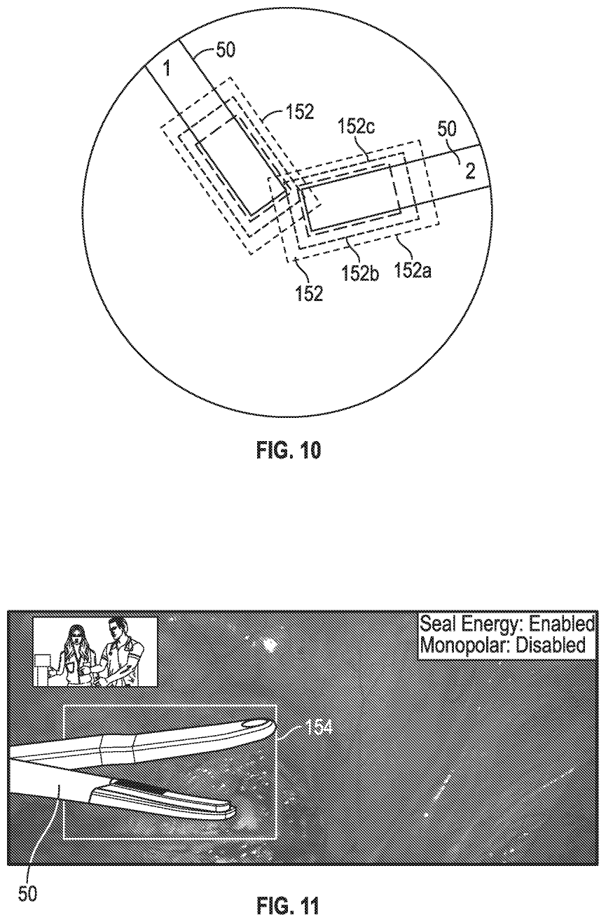

At step 104, the video processing device 56 determines whether a plurality (i.e., two or more) of the instruments 50 are present in a field of view of the camera 51, i.e., visible to the camera 51, using a computer vision algorithm for identifying the instruments 50 and/or fiducial markers 150 (e.g., see FIG. 10). If only one and/or an insufficient amount of the instruments 50 are captured by the camera 51, then at least one of the instruments 50 or the camera 51 are repositioned by the corresponding robotic arms 40 at step 106 and the method is repeated to verify whether the instruments 50 are visible to the camera 51.

If multiple instruments 50 are visible, then at step 108 the controller 21a determines initial position of the instruments 50 using the fiducial markers 150. At step 110, the controller 21 a tracks subsequent movement of the instruments 50 using kinematics data (e.g., angles of the links 42a, 42b, 42c and position of the joints 44a, 44b, 44c of the robotic arm 40) based on the initial position determined at step 208. At step 112, controller 21a performs a check whether any of the robotic arms 40 controlling the instruments 50 and/or the camera 51 has been moved as that would result in shifting the initial position and the subsequent tracking. Determination that the arms have been manually moved may be determined based on torque, IMU, or other sensors of the robotic arm 40 and/or the setup arm 61 configured to detect external impacts. If the robotic arms 40 have been moved, then the method proceeds back to step 102 to receive the video stream repeat the steps 102-112. If the robotic arms 40 have not been manually moved, then the method proceeds to step 114 to monitor collisions between the instruments 50 and/or robotic arms 40.

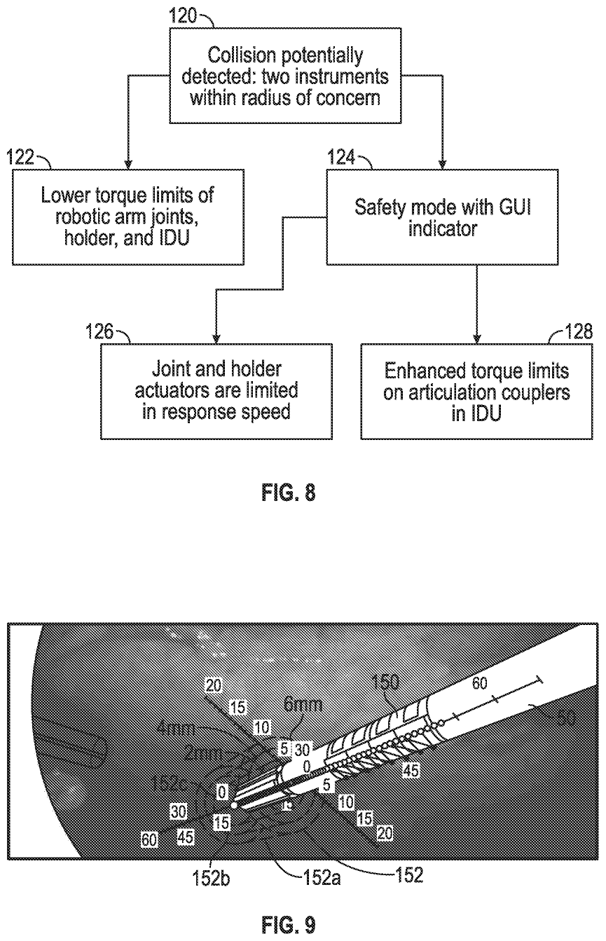

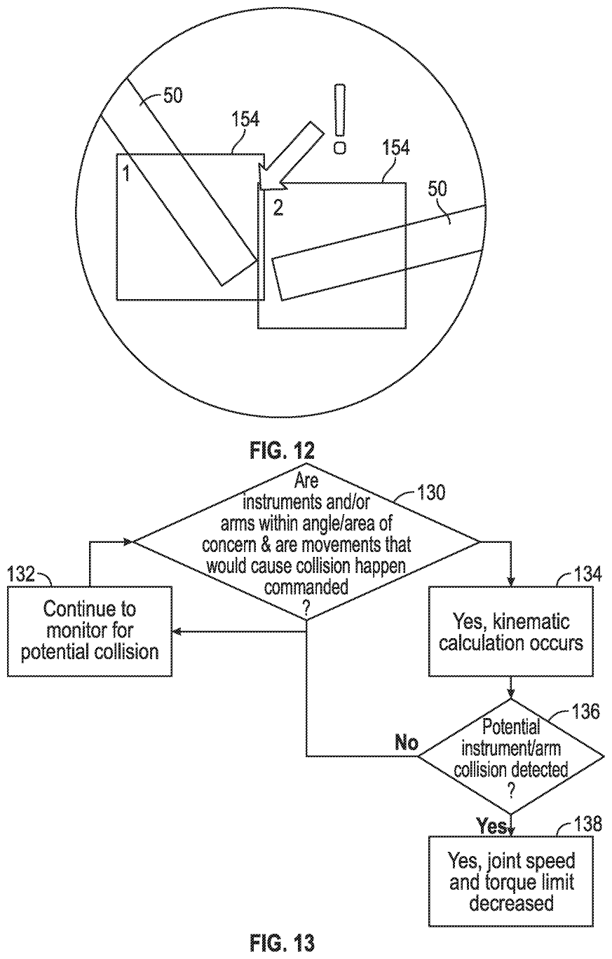

With reference to FIG. 8, a method for predicting and responding to potential instrument collisions includes at step 120 detecting a potential collision between two instruments 50. Potential collision is detected based on virtual boundary zones, which may be 3D shapes (e.g., FIGS. 9 and 10) or 2D shapes (e.g., FIGS. 11 and 12). With reference to FIGS. 9 and 10, a 3D boundary volume 152 is generated by the video processing device 56 around any portion of the instrument 50, e.g., end effector, jaws, etc. The boundary volume 152 may be any suitable shape, e.g., cylindrical (as shown), spherical, polygonal prism, and combination thereof. The boundary volume 152 may include a plurality of nested volumes 152a-c, providing different degrees or buffers for detecting potential collisions. The boundary volume 152 may be displayed on the video feed as shown in FIG. 9 or may be hidden from view. Each of the instruments 50 includes a boundary volume 152, each of which is used to detect potential collisions by the controller 21a. The potential collision is detected based on overlap of boundary volumes 152 as shown in FIG. 10, i.e., if there is overlap then controller 21a determines that the instruments 50 may collide.

With reference to FIGS. 11 and 12, a 2D shape 154 may also be generated by the video processing device 56 around the end effector of the instrument 50. The shape 154 may be any suitable shape, e.g., polygon, circle, and combination thereof. Shape 154 may include a plurality of nested shapes, providing different buffer zones for detecting potential collisions. Shape 154 may be displayed on the video feed as shown in FIG. 11 or may be hidden from view. Each of the instruments 50 includes a shape 154, both of which are used to detect potential collisions by the controller 21a. The potential collision is detected based on overlap of shapes 154 as shown in FIG. 12, i.e., if there is overlap then controller 21a determines that the instruments 50 may collide.

The potential collision is monitored continuously during operation of the robotic system 10, if a potential collision is detected, then the controller 21a proceeds to one or both of the steps 122 and 124. At step 122, the controller 21a lowers torque limits of the actuators in the joints 44a, 44b, 44c, holder 46 of the robotic arm 40 and the IDU 52. The torque limits prevent the actuators from operating at full torque, thus preventing damage to the instruments 50 if they were indeed to collide. At step 124, the controller 21a also outputs GUI indicators or prompts on the screens 32, 34, and/or 23 informing of the potential collision. The prompts may be based on the proximity of the instruments 50 and/or the degree of overlap and/or which of the nested boundaries are overlapping. Thus, different types of prompts are output based on the likelihood of the collision. The prompts may be color coded, e.g., green for slight overlap of the boundary volumes or shapes, yellow for medium overlap, and red for extreme overlap or almost touching of the instrument. The nested volumes 152a-c may change color to illustrate the severity of the overlap as shown in FIG. 10 or warning prompts, e.g., exclamation marks, may be displayed as shown in FIG. 12. The user may dismiss the prompts depending on severity of the warning, i.e., medium or extreme warnings cannot be dismissed, whereas warning regarding slight overlapping may be dismissed.

In addition to the GUI indicators, controller 21a also performs additional safety processes in steps 126 and 128. At step 126, controller 21a sets speed limits for the actuators in the joints 44a, 44b, 44c, holder 46 of the robotic arm 40 and the IDU 52. At step 128, the controller 21a may further lower the torque limits as described in step 122. The limits may be set based on the degree of overlap. Similarly to the different degrees of warnings and GUI indicators being output based on the degree of overlap of the boundary volumes/shapes, the torque limits may be similarly tailored to the overlap. The same tailoring mechanic may be used with speed limits.

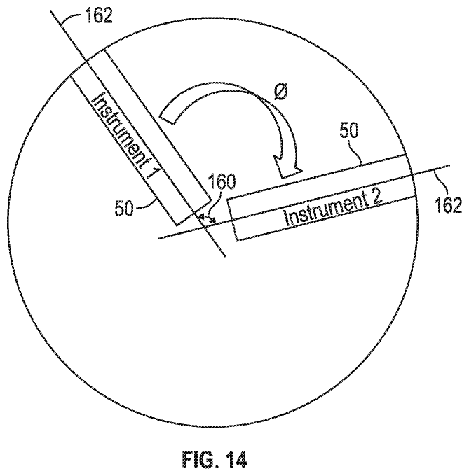

FIG. 13 shows a flow chart of another method for predicting and responding to instrument collisions, and includes at step 130 detecting a potential collision between two instruments 50 and/or robotic arms 40. With reference to FIG. 14, for the instruments 50, potential collision may be detected based on measuring an angle 160 between longitudinal axes 162 defined by the instruments 50, which is also indicative of the position of the robotic arms 40 (i.e., since the instruments 50 are aligned the same axis as the holder 46 of the robotic arms 40). The angle is determined by the by the video processing device 56, which initially identifies the instruments 50 in the video stream, then defines the longitudinal axis 162 through each of the instruments 50, and then calculates the angle 160. The controller 21a may also calculate the angle 160 from the longitudinal axes 162 provided by the video processing device 56. The controller 21a then compares the angle 160 to a threshold indicative of a potential collision, e.g., 30°. If the calculated angle is above the threshold, which is indicative of no potential collision, then the controller 21a continues to monitor for potential collisions at step 132.

Monitoring collisions of robotic arms 40 may be performed using an external vision tracking unit 170 (FIG. 1) having one or more cameras or infrared sensors or other imaging devices configured to track the locations of the robotic arms 40, which may have a marker 172 disposed thereon. The tracking unit 170 may be disposed on the control tower 20 or any other location in the operating room. The tracking unit 170 may include one or more position sensors, which may be any suitable white light or infrared cameras, electromagnetic sensors, magnetoresistance sensors, radio frequency sensors, or any other sensor adapted to sufficiently sense the position of a navigation marker. The tracking unit 170 may be configured to detect markers 172 disposed on the robotic arm 40, i.e., the holder 46. The markers 172 may be passive tracking elements (e.g., reflectors) for transmitting light signals (e.g., reflecting light emitted from the tracking unit 170). Alternatively, the markers 172 may include a radio opaque material that is identified and trackable by the tracking unit 170. In other configurations, active tracking markers can be employed. The active tracking markers can be, for example, light emitting diodes transmitting light, such as infrared light. Active and passive arrangements are possible. The markers may be arranged in a defined or known position and orientation relative to the other markers in order to allow the tracking unit 170 to determine the position of the robotic arms 40, and in particular the holders 46, relative to each other. Tracking the position and orientation of the holders 46 allows the surgical robotic system 10 to determine the position and/or orientation of the instruments 50 and the camera 51 within a defined space, such as the surgical field.

For the robotic arms 40, potential collision is determined based on the distance between the markers 172. The tracking unit 170 provides the distance measurement to the controller 21a, which then compares the measured distance to a threshold, which may be from about 6 inches to about 24 inches. If the measured distance is above the threshold, which is indicative of no potential collision, then the controller 21a continues to monitor for potential collisions at step 132.

If the angle thresholds for the instruments 50 and/or the distance between markers 172 are crossed, i.e., measured angle and distance are lower than the threshold, then the controller 21a indicates that a potential collision between the instruments 50 and/or the robotic arms 40 is possible. Warning prompts of the potential collision may be displayed as described above with respect to the method of FIG. 8.

At step 134, the controller 21a calculates if a commanded movement will result in a collision, i.e., once the instruments 50 and/or the robotic arm 40 are in a potential collision configuration execution of the movement command will result in a collision. The controller 21a performs kinematic calculations based on a received input command to confirm whether an actual collision may result in response to moving the robotic arms 40 to achieve the commanded pose from the point where the potential collision was detected. Thus, kinematic calculations are used to determine the position and orientation of the robotic arm 40 along with the tracking data from the tracking unit 170.

At step 136, the controller 21a predicts whether an actual collision is about to occur by analyzing kinematic calculations for each of the robotic arms 40 and/or instrument 50 indicating the final pose, to determine if there is any overlap between the kinematic solutions for different arms 40 and/or instrument 50, which would indicate a collision. If so, then at step 138, the controller 21a lowers torque and speed limits of the actuators in the joints 44a, 44b, 44c, holder 46 of the robotic arm 40 and the IDU 52. The torque and speed limits prevent the actuators from operating at full torque, thus preventing damage to the instruments 50 if they were indeed to collide. If no collision is detected, then the controller proceeds to step 132 to continue to monitor for potential collisions.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a first robotic arm including a first robotic instrument;
a second robotic arm including a second robotic instrument;
a camera configured to capture a video stream of the first robotic instrument and the second robotic instrument;
a video processing device coupled to the camera and configured to:
identify the first robotic instrument and the second robotic instrument in at least one frame of the video stream;
generate a first boundary around a portion of the first robotic instrument;
generate a second boundary around a portion of the second robotic instrument; and
calculate an overlap between the first boundary and the second boundary; and
a controller configured to:
predict a collision based on the overlap between the first boundary and the second boundary; and
adjust at least one parameter of the first robotic arm or the second robotic arm in response to prediction of the collision.

2. The surgical robotic system according to claim 1, wherein each of the first boundary and the second boundary is a 3D boundary volume.

3. The surgical robotic system according to claim 2, wherein the 3D boundary volume includes a plurality of nested volumes.

4. The surgical robotic system according to claim 1, where each of the first boundary and the second boundary is a 2D shape.

5. The surgical robotic system according to claim 1, wherein the at least one parameter is selected from the group consisting of a torque limit or a speed limit of an actuator of the first robotic arm or the second robotic arm.

6. The surgical robotic system according to claim 1, further comprising a screen configured to display a graphical user interface (GUI).

7. The surgical robotic system according to claim 6, wherein the controller is further configured to output a prompt on the GUI in response to the prediction of the collision.

8. A surgical robotic system comprising:
a first robotic arm including a first robotic instrument;
a second robotic arm including a second robotic instrument;
a camera configured to capture a video stream of the first robotic instrument and the second robotic instrument;
a video processing device coupled to the camera and configured to:
identify the first robotic instrument and the second robotic instrument in at least one frame of the video stream; and
calculate an angle between the first robotic instrument and the second robotic instrument; and
a controller configured to:
compare the calculated angle to an angle threshold;
predict a collision based on the calculated angle being lower than the angle threshold; and
adjust at least one parameter of the first robotic arm or the second robotic arm in response to prediction of the collision.

9. The surgical robotic system according to claim 8, wherein the first robotic arm includes a first marker and the second robotic arm includes a second marker.

10. The surgical robotic system according to claim 9, further comprising:
a tracking unit configured to detect locations of the first marker and the second marker.

11. The surgical robotic system according to claim 10, wherein the controller is further configured to calculate a distance between the first robotic arm and the second robotic arm based on the locations of the first marker and the second marker.

12. The surgical robotic system according to claim 11, wherein the controller is further configured to compare the calculated distance to a distance threshold.

13. The surgical robotic system according to claim 12, wherein the controller is further configured to predict the collision based on the calculated distance being lower than the distance threshold.

14. The surgical robotic system according to claim 13, further comprising a surgeon console including at least one hand controller configured to receive user input for controlling at least one of the first robotic arm or the second robotic arm.

15. The surgical robotic system according to claim 14, wherein the controller is further configured to calculate a pose for each of the first robotic arm and the second robotic arm based on the user input.

16. The surgical robotic system according to claim 14, wherein the controller is further configured to determine whether the calculated pose of the first robotic arm and the second robotic arm results in a collision.

17. The surgical robotic system according to claim 8, further comprising a screen configured to display a graphical user interface (GUI).

18. The surgical robotic system according to claim 17, wherein the controller is further configured to output a prompt on the GUI in response to prediction of the collision.

19. A method for detecting a potential collision between robotic instruments, the method comprising:
capturing through a laparoscopic camera a video stream of a first robotic instrument of a first robotic arm and a second robotic instrument of a second robotic arm;
identifying, at a video processing device, the first robotic instrument and the second robotic instrument in at least one frame of the video stream;
generating a first boundary around a portion of the first robotic instrument;
generating a second boundary around a portion of the second robotic instrument; and
calculating an overlap between the first boundary and the second boundary; and
predicting a collision based on the overlap between the first boundary and the second boundary; and
adjusting at least one parameter of the first robotic arm or the second robotic arm in response to prediction of the collision. The method according to claim 19, further comprising:
displaying, on a screen, a graphical user interface (GUI); and
outputting a prompt on the GUI in response to prediction of the collision.
